SEQUENCE LISTING (15 sequences)

SEQ ID NO: 1 - protein, 7 aa, Homo sapiens
TEEENLR

SEQ ID NO: 2 - protein, 11 aa, Homo sapiens
TEEENLRKGGK

SEQ ID NO: 3 - protein, 14 aa, Homo sapiens
KKPLDGEYFTLQIR

SEQ ID NO: 4 - protein, 13 aa, Homo sapiens
EPGGSRAHSSHLK

SEQ ID NO: 5 - protein, 27 aa, Homo sapiens
GEPHHELPPGSTKRALPNNTSSSPQPK

SEQ ID NO: 6 - protein, 572 aa, Homo sapiens
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 7 - protein, 214 aa, Homo sapiens
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 8 - protein, 117 aa, Homo sapiens
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARGGYYAMDYWGQGTSVTVSS

SEQ ID NO: 9 - protein, 107 aa, Homo sapiens
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPYTFGGGTKLEIK

SEQ ID NO: 10 - protein, 5 aa, Homo sapiens
SYVMH

SEQ ID NO: 11 - protein, 17 aa, Homo sapiens
YINPYNDGTKYNEKFKG

SEQ ID NO: 12 - protein, 8 aa, Homo sapiens
GGYYAMDY

SEQ ID NO: 13 - protein, 11 aa, Homo sapiens
RASQDISNYLN

SEQ ID NO: 14 - protein, 7 aa, Homo sapiens
YTSRLHS

SEQ ID NO: 15 - protein, 9 aa, Homo sapiens
QQGNTLPYT

(19) United States
(12) Patent Application Publication
(10) Pub. No.: US 2024/0060994 A1
(43) Pub. Date: Feb. 22, 2024

(54) p53 Peptides as Markers in the Diagnosis and Prognosis of Alzheimer's Disease

(71) Applicant: Diadem SpA, Milan (IT)

(72) Inventors: Daniela Letizia Uberti, Brescia (IT); Maurizio Memo, Monticelli Brusati (IT)

(21) Appl. No.: 18/300,535

(22) Filed: Apr. 14, 2023

Related U.S. Application Data
(63) Continuation of application No. 17/113,559, filed on Dec. 7, 2020, now Pat. No. 11,650,215.
Application No. 17/113,559 is a continuation of application No. PCT/IB2019/051785, filed on Mar. 6, 2019.

(51) Int. Cl. G01N 33/68

(52) U.S. Cl. CPC G01N 33/6896; G01N 2800/2821; G01N 2800/52

(57) ABSTRACT

Disclosed are p53 peptides and their use as biomarkers in the diagnosis and/or prognosis of Alzheimer's disease (AD) in a biological sample. The invention also provides for a diagnostic method based on a highly accurate mass spectrometry analysis for the diagnosis of Alzheimer's disease at the pre-clinical and prodromal stages of the disease and for the prognosis of cognitive decline in a subject, by quantitating the levels of said p53 peptides specifically in human plasma of patients.

### CROSS-REFERENCE TO RELATED APPLICATIONS

This U.S. patent application is a continuation of U.S. patent application Ser. No. 17/113,559, filed Dec. 7, 2020, now U.S. Pat. No. 11,650,215, which is a continuation of International Patent Application No. PCT/IB2019/051785, filed Mar. 6, 2019, the contents of each of which is herein incorporated by reference in its entirety.

### SEQUENCE LISTING

This application contains a Sequence Listing which has been submitted electronically as a WIPO Standard ST.26 XML file via Patent Center, created on Nov. 13, 2023, is entitled “121689-10203.xml” and is 14.6 KB in size. The sequence listing is incorporated herein by reference in its entirety.

### FIELD OF THE INVENTION

The present invention refers to p53 peptides P1, P2, P3 and P5 and to their use as biomarkers in the diagnosis and/or prognosis of Alzheimer's disease (AD) in a biological sample. The invention also provides for a diagnostic method based on a highly accurate mass spectrometry analysis for the diagnosis of Alzheimer's disease at the pre-clinical and prodromal stages of the disease and for the prognosis of cognitive decline in a subject, by quantitating the levels of said p53 peptides specifically in human plasma of patients.

### BACKGROUND ART

The confirmation of the presence of a large amount of altered conformational p53 isoform as an early risk factor for Alzheimer's disease (shortly ‘AD’) have been demonstrated in different published studies [1-3]. Initially, more than 400 subjects among AD, Mild Cognitive Impairment, Parkinson Disease, other Dementia and healthy subjects were enrolled in different independent studies and tested for Unfolded p53 by using different techniques (immunoprecipitation experiments, FACS analysis, ELISA) with a commercial conformational specific anti-p53 antibody [4-7]. In 2006 for the first time Uberti et al. [8], demonstrated that fibroblasts from sporadic Alzheimer's disease (AD) patients specifically expressed an anomalous and detectable conformational state of p53 that differentiate these cells from fibroblasts of age-matched non-AD subjects. In this conformational altered state, p53 lost its ability to transactivate its target genes, and consequently its biological functions [9-10]. The higher amount of unfolded p53 was also confirmed in blood of AD compared to healthy-non demented subjects or patients affected by other dementia and PD, as well as in MCI converted to AD.

Altogether these data suggested a direct association between Unfolded p53 and AD pathology.

In EP3201234B1, it has been reported the development of a new conformational specific anti-Up53 antibody named 2D3A8, that binds to an epitope (aa 282-297), accessible only when p53 loses its wild type conformation towards an unfolded phenotype. Comparing to the commercial antibody used at the beginning of Unfolded p53 discovering in AD (PAb240, aa214-217), the 2D3A8 antibody showed higher sensitivity and specificity in identifying AD patients compared to healthy elderly in Oviedo cohort.

In particular, said immunodiagnostic method is able to identify immunocomplex in a biological sample that are indicative of AD and to determine the predisposition of a subject affected by Mild Cognitive Impairment (MCI) to develop AD.

There is now the need of identifying new specific biological markers that can be used in the diagnosis and/or prognosis of Alzheimer's disease and of developing an accurate and sensible diagnostic method that can be used for the diagnosis and/or prognosis of AD, in particular at the pre-clinical and prodromal stages of the disease and for the differential analysis of AD from other forms of dementia.

### SUMMARY OF THE INVENTION

This object has been achieved by identifying four p53 peptides, called P1 (SEQ ID N. 1), P2 (SEQ ID N. 2), P3 (SEQ ID N. 3) and P5 (SEQ ID N. 5) having the sequences reported in the claims, in a biological sample.

Another aspect of the present invention relates to a diagnostic method based on the identification and quantification of said peptides, for use in the diagnosis and/or in the prognosis of Alzheimer's disease at different stages.

The characteristics and the advantages of the present invention will become apparent from the following detailed description and the working examples provided for illustrative purposes.

### DETAILED DESCRIPTION OF THE INVENTION

The invention therefore relates to a highly accurate mass spectrometry method for the diagnosis of Alzheimer's disease at the pre-clinical and prodromal stages of the disease. Said method is based on the identification and quantification of the levels of specific p53 peptides, shorty referred to as “P1”, “P2”, “P3”, and “P5” or “p53 peptides”, that have been detected by mass spectrometry analysis in human plasma of patients affected by Alzheimer's disease or patients that have symptoms that can predispose to the development of AD.

In particular, the mass spectrometry analysis is a qualitative and quantitative method that was performed when no information about the exact correlation between these specific sequences of p53 and AD was known.

First, p53 peptides, present in plasma of patients affected by AD, have been identified by deep sequencing protein method in plasma from patient at pre-clinical, prodromal clinical stages of Alzheimer's, MCI stable patients, and cognitive normal subjects. Then the levels of the p53 peptides have been quantitated in plasma with a highly sensitive selective reaction monitoring (SRM) mass spectrometry method carrying out an Area Under the ROC Curve (AUC) (where ‘ROC Curve’ means ‘receiver operating characteristic curve’).

The data obtained therefore demonstrate a strong evidence that the p53 peptides can be considered as biomarkers in the diagnosis and/or prognosis of AD.

Said method is advantageously fast, requires a small volume of plasma sample and quantifies the concentration of the p53 peptides in each sample analysed.

Furthermore, the method and the biomarkers identified can be used also in the diagnosis of Alzheimer's disease in asymptomatic individuals and people suffering from MCI, allowing the access to the diagnostics market.

In addition, since said biomarkers can be used in the prognosis of cognitive decline to Alzheimer's Dementia in asymptomatic and MCI subjects, said method allows the use of a p53 peptide expression to select the subjects in clinical trials to enable success of the trial and to differentiate patients affected by AD from other forms of dementia.

It is therefore an embodiment of the present invention a p53 peptide consisting of formula (I) P1: TEEENLR (SEQ ID N. 1). Alternatively, the p53 peptides of the present invention have an amino acid sequence with at least 80-90% of identity to the sequence of formula (I), preferably at least 90-95% of identity to the sequence of formula (I), more preferably at least 96-99% of identity to the sequence of formula (I).

A further embodiment of the present invention is a p53 peptide having formula (II) P2: TEEENLRK[GG]K (SEQ ID N. 2), where [GG] is a moiety of a residue tail of ubiquitin that derives from the ubiquitination at K291 of the p53 protein after post-translational modification. In this regard, for the purposes of the present invention, the term in square brackets “[GG]” denotes that the first-K amino acid of the P2 sequence is branched by said moiety.

An additional embodiment of the present invention is a p53 peptide consisting of formula (II) P2: TEEENLRK[GG]K (SEQ ID N. 2), where [GG] is as above defined. Alternatively, the p53 peptides of the present invention have an amino acid sequence with at least 80-90% of identity to the sequence of formula (II), preferably at least 90-95% of identity to the sequence of formula (II), more preferably at least 96-99% of identity to the sequence of formula (II).

A further embodiment is the use of the p53 peptides P1 and/or P2 according to the present invention as in vitro biomarker for the diagnosis and/or prognosis of Alzheimer's disease.

A further embodiment of the present invention is a p53 peptide having formula (III) P3: KKPLDGEYFTLQIR (SEQ ID N. 3).

An additional embodiment of the present invention is a p53 peptide consisting of formula (III) P3: KKPLDGEYFTLQIR (SEQ ID N. 3).

A further embodiment of the present invention is a p53 peptide having formula (V) P5: GEPHHELPPGSTKRALPNNTSSSPQPK (SEQ ID N. 5).

An additional embodiment of the present invention is a p53 peptide consisting of (V) P5: GEPHHELPPGSTKRALPNNTSSSPQPK (SEQ ID N. 5).

A further embodiment is the use of the p53 peptides P3 and/or P5 according to the present invention as in vitro biomarker for the diagnosis of Alzheimer's disease.

A further embodiment of the present invention is an in vitro or ex vivo method for the diagnosis and/or prognosis of Alzheimer's disease, the method comprising the steps of:
a) determining the presence of a p53 peptide in a biological sample, and
b) quantifying said peptide by comparing the same with a given internal control.

In a preferred embodiment, the p53 peptide in step a) is the p53 peptide P1 or P2.

In a preferred embodiment, the method of the present invention also comprises a step c) of correlating the quantity of the p53 peptide with the diagnosis of Alzheimer's disease in a patient at different stages of the diseases.

Preferably, said biological sample is blood, plasma, serum, saliva, urine, neuronal cells, blood cells or other types of cells.

In a preferred embodiment, in the method according to the present invention, the step a) is performed by immunoprecipitating the p53 peptide with a monoclonal antibody that binds to a p53 peptide.

Preferably, said monoclonal antibody is the antibody 2D3A8. The amino acid sequences of the 2D3A8 antibody include the heavy chain (SEQ ID NO: 6) and light chain (SEQ ID NO: 7), heavy chain variable region (SEQ ID NO: 8) and light chain variable region (SEQ ID NO: 9), heavy chain CDRs 1, 2 and 3 (SEQ ID NOS: 10, 11 and 12, respectively) and light chain CDRs 1, 2 and 3 (SEQ ID NOS: 13, 14 and 15, respectively).

In a preferred embodiment in the method according to the present invention the step b) is performed through mass spectrometry analysis, preferably by HPLC-mass spectrometry.

In a further preferred embodiment in step b) labelled peptides are used as internal controls.

According to a preferred embodiment the in vitro or ex vivo method of the present invention comprises the following steps:
a) determining the presence of the p53 peptide in a biological sample, by:
   (i) providing a biological sample;
   (ii) performing protein immunoprecipitation by an antibody that binds a p53 peptide;
   (iii) performing protein fragmentation by trypsin;
   (iv) performing Strong Cation Exchange Chromatography of the peptides;
and
b) quantifying said p53 peptide by comparing the same with a given internal control, by:
   (v) performing a Selected Reaction Monitoring analysis to identify and quantify the p53 peptide by comparing it with a given control.

Preferably, the antibody of step a) (ii) is 2D3A8.

Preferably, the biological sample of step a)(i) is subjected to protein plasma depletion by HPLC or chromatographic columns, before performing step a)(ii).

According to a preferred embodiment the in vitro or ex vivo method of the present invention is used for the diagnosis of Alzheimer's disease in asymptomatic individuals and people suffering from MCI.

Preferably, in asymptomatic individuals and people suffering from MCI, the quantity of the p53 peptide is of 0.05 fmol/40 μl to 6.70 fmol/40 μl.

According to a further preferred embodiment, the in vitro or ex vivo method of the present invention is used for the prognosis of cognitive decline of Alzheimer's disease in asymptomatic individuals and people suffering from MCI.

Preferably, in asymptomatic and MCI subjects that have the prognosis of cognitive decline of Alzheimer's dementia, the quantity of the p53 peptide is of 0.203 fmol/40 μl to 6.70 fmol/40 μl.

Preferably, in asymptomatic individuals, the AUC is at least 80%.

Preferably, in people suffering from MCI, the AUC is at least 90%.

Measures of accuracy of a diagnostic/prognostic method include sensitivity and specificity.

Sensitivity is the probability of a positive test result among those having the target condition, and it is measured by the formula: Sensitivity=true positives/(true positive+false negative).

Specificity is the probability of a negative test result among those without the target condition, and it is measured by the formula: Specificity=true negatives/(true negative+false positives).

Preferably, in asymptomatic individuals, the sensitivity of the p53 peptide is at least 70%.

Preferably, in people suffering from MCI, the sensitivity of the p53 peptide is at least 90%.

Preferably, in asymptomatic individuals, the specificity of the p53 peptide is at least 90%.

Preferably, in people suffering from MCI, the specificity of the p53 peptide is at least 90%.

In a further preferred embodiment, the in vitro or ex vivo method of the present invention is used for the differential analysis of Alzheimer's disease from other dementia, said other dementia being selected from fronto temporal dementia (FTD), Lewi's Body, Parkinson's disease and vascular dementia.

Preferably, in patients affected by Alzheimer's disease, the quantity of said p53 peptide is of 2.21 fmol/40 μl to 6.56 fmol/40 μl, while in patients affected by other forms of dementia the quantity of said p53 is lower than 3.81 fmol/40 μl.

Preferably, in patients affected by Alzheimer's disease, the AUC is at least 85%.

Preferably, in patients affected by Alzheimer's disease, the sensitivity of the p53 peptide is at least 75%.

Preferably, in patients affected by Alzheimer's disease, the specificity of the p53 peptide is at least 80%.

A further embodiment of the present invention is a method of detecting a p53 peptide as above described, said method comprising the following steps:
a) determining the presence of the p53 peptide in a biological sample, and
b) quantifying said peptide by comparing the same with a given control.

In a further preferred embodiment, the method of the present invention further comprising a step c) of correlating the quantity of the p53 peptide with the diagnosis and/or prognosis of Alzheimer's disease.

According to a preferred embodiment, in the method of the present invention, step c) correlates the quantity of the p53 peptide with the diagnosis of Alzheimer's disease in asymptomatic individuals and people suffering from MCI.

In a preferred embodiment, the p53 peptide in step a) is the p53 peptide P1 or P2.

According to a further preferred embodiment, in the method of the present invention, in step c) the quantity of the p53 peptide is correlated with the prognosis of cognitive decline of Alzheimer's disease in asymptomatic individuals and people suffering from MCI.

More preferably, in the method of the present invention, in step c) the quantity of the p53 peptide is correlated with the differential analysis of Alzheimer's disease from other dementia, said other dementia being selected from fronto temporal dementia (FTD), Lewi's Body, Parkinson's disease and vascular dementia.

It should be also understood that all the combinations of preferred aspects of the peptides of the invention, as well as of the preparation processes, and methods using of the same, as above reported, are to be deemed as hereby disclosed.

All combinations of the preferred aspects of the peptides of the invention, preparation processes, and methods disclosed above are to be understood as herein described.

Below are working examples of the present invention provided for illustrative purposes.

### Examples

### Materials and Methods

### Materials

Antibody-based plasma/serum depletion Seppro® IgY14 LC10 column was purchased from Sigma-Aldrich (Merck KGaA, Darmstadt, Germany). Trypsin (Sequencing grade modified porcine) was obtained from Promega. Acetonitrile was purchased from JT Baker, and formic acid was obtained from EMD Millipore (Billerica, MA, USA). C18 Cartridges for sample preparation, and chromatography columns for bRPLC and online HPLC of Triple Quadrupole mass spectrometer were purchased from Waters (Milford, Massachusetts). Plasma Dilution Buffer, -PlasmaWashing Buffer and -Plasma Elution Buffer, were prepared following standard protocols for this method. All other reagents were purchased from Sigma-Aldrich (St. Louis, Missouri) unless otherwise indicated.

### Plasma Sample Depletion

The most abundant proteins in the plasma were depleted using a Seppro IgY14 column. Plasma samples were diluted 5× in Plasma Dilution buffer, filtered (0.22 m), then injected into IgY LC10 columns attached to an Agilent 1200 HPLC system. The unretained fraction was collected. High-abundance proteins were eluted Plasma Elution buffer.

### Immunoprecipitation (IP)

Antibody clone 2D3A8 was used in the IP reactions. Conjugation of antibodies to beads was optimized and performed using with Link buffers. The monoclonal antibody was added directly to Protein G Dynal Magnetic Beads (as obtained from Invitrogen), and the antibody was bound to the beads in a buffer on a rotator at room temperature for 2 h. The antibody-bound beads then were washed by incubation in 50 mL Link-A buffer and collected on a magnet. The antibody was cross-linked to the protein G on the beads by incubation with 50 mL B buffer on a rotator at room temperature for 1 h. The beads then were washed twice with 50 mL C buffer, resuspended in 50 mL C buffer, and rotated at room temperature for 15 min. The beads were resuspended in C buffer and were stored at −20° C. until application. 200 μl depleted or undepleted plasma samples were used for IP reactions, each sample is reconstituted with IP buffer into a 3 ml system and 3 ml antibody conjugated beads (1:1 volume ratio) were added to the system. The IP systems were incubated on a rotator with a speed of 32 rounds per minute at 4° C. for 18 hours. Beads were collected on a magnet and washed by IP-Wash buffer at 4° C. for 3 times, followed by elution of the antigen through IP-Elution buffer. Flow-through fractions were collected for protein sample preparation.

### Protein Preparation

Protein samples were processed in compliance with “Filter Assisted Sample Preparation” (FASP) method (NatMethods. 2009, 6:359-362). Briefly, protein samples in 9M UREA were reduced with 5 mM TCEP at 37° C. for 45 min and reduced cysteines were blocked using 50 mM iodoacetamide at 25° C. for 15 min. Protein samples (100 μg each) were then cleaned using 10 kDa Amicon Filter (UFC501096, Millipore) three times using 9M urea and two times using Buffer 1. Samples were then proteolyzed with trypsin (V5111, Promega) for 12 hrs at 37° C. The peptide solution then was acidified by adding 1% trifluoroacetic acid (TFA) and was incubated at room temperature for 15 min. A Sep-Pak light C18 cartridge (Waters Corporation) was activated by loading 5 mL 100% (vol/vol) acetonitrile and was washed by 3.5 mL 0.1% TFA solution two times.

Acidified digested peptide solution was centrifuged at 1,800×g for 5 min, and the supernatant was loaded into the cartridge. To desalt the peptides bound to the cartridge, 1 mL, 3 mL, and 4 mL of 0.1% TFA were used sequentially. To elute the peptides from the cartridge, 2 mL of 40% (vol/vol) acetonitrile with 0.1% TFA was used, and this elution was repeated two more times (for a total of 6 mL of eluate). It was important to ensure that the cartridge had stopped dripping before each sequential wash and elution solution was applied. The eluted peptides were lyophilized overnight and reconstituted in 100 μL of Buffer 2.

### Strong Cation Exchange Chromatography

Peptides were fractionated by strong cation exchange (SCX) chromatography. Briefly, lyophilized peptides mixture was resuspended in 1 ml of SCX solvent A (5 mM KH2PO4 pH 2.7, 30% ACN) and fractionated by SCX chromatography on a PolySULPHOETHYL A (5 m, 200 Å) column (200×9.4 mm; PolyLC Inc., Columbia, MD) by employing an increasing gradient of SCX solvent B (5 mM KH2PO4 pH 2.7, 30% ACN, 350 mM KCl) on an Agilent 1290 Infinity II LC system. For each experiment, a total of 96 fractions were initially collected, which were then pooled into 24 fractions, and dried using SpeedVac (Eppendorf). bRPLC and SCX were performed side by side to compare the compatibility of detection of TP53 proteins, and SCX was chosen because a relative stronger yield of TP53 isoforms has been repeatedly observed for this project.

### Optimization of Peptide SRM Transitions

4 peptides from targeting TP53 protein were chosen as SRM quantifying targets and 1092 sets of transition parameters and retention times of the 4 peptides were established individually using an Agilent 6495 Triple Quadrapole Mass Spectrometer for 1+, 2+, 3+ and 4+ charged precursor ions. 2 peptides (P1 and P2) were then selected as main correlated to the AD pathology.

### Selected Reaction Monitoring (SRM) Analysis

Peptide samples reconstituted in 37 ul Buffer 3 were spiked with SRM Internal Control Mixture composed of a pool of 1 femto mole heavy isotope labeled peptides covering a large hydrophobicity window and a large M/z range (M/z 200˜ 1300), Peptide samples were eluted through an online Agilent 1290 HPLC system into the Jet Stream ESI source of an Agilent 6495 Triple Quadrupole Mass spectrometer.

### Results

There were cumulatively 5 peptides detected in previous projects for p53 protein in plasma [P1: TEEENLR (SEQ ID N. 1); P2: TEEENLRK[GG]K (SEQ ID N. 2), P3: KKPLDGEYFTLQIR (SEQ ID N. 3), P4: EPGGSRAHSSHLK (SEQ ID N. 4); and P5: GEPHHELPPGSTKRALPNNTSSSPQPK (SEQ ID N. 5)]. It was established the SRM detection method for 4 peptides. The peptide GEPHHELPPGSTKRALPNNTSSSPQPK (SEQ ID N. 5) is not feasible for SRM-based detection due to its high molecular weight.

Correlation between p53 sequence peptide and AD diagnosis.

### Example 1. Detection of p53 Peptide Markers for Alzheimer's at the Asymptomatic (Cognitive Normal) and MCI Stages by Deep Sequencing Protein Method

A deep sequence mass spectrometry analysis of human plasma immuno-precipitated was conducted with the conformationally specific Ab 2D3A8. Plasma sample was depleted from abundant proteins according to the method reported above. Samples were then immunoprecipitated by 2D3A8 and analyzed using Orbitrap (deep sequence mass spectrometry analysis). The resulting peptides present uniquely in AD, MCI to AD and asymptomatic to AD and absent in asymptomatic and MCI stable samples were chosen as markers. The detection of the peptide (presence/absence) by this method was correlated with clinical diagnosis of the subjects.

It was analyzed a pool of 10 samples from PiB (Pittsburgh compound B)+ve AD (PiB+ve) patients (AD), a pool of 10 baseline plasma samples from asymptomatic PiB negative individuals who remained both asymptomatic and PiB-ve for at least 108 months post baseline (Normal), a pool of 10 baseline plasma samples from PiB+ve patients with MCI 18 months before they converted to AD symptoms (Prodromal, MCI due to AD), a pool of 10 baseline plasma samples from PiB-ve patients with MCI who did not experience further cognitive decline to AD symptoms (follow up 18-72 months post baseline), (MCI stable) and 3 baseline plasma samples from PiB+ve patients with MCI 18 months before they converted to AD symptoms (Preclinical, Normal to AD converters).

TABLE 1
Inclusion Criteria for baseline characteristics

| | Minimum age | f/m (minimum %) |
|---|---|---|
| Prodromal (MCI to AD) | 70 | 0.40 |
| Preclinical (Cognitive Normal to AD | 70 | 0.4 |
| converters) | | |
| MCI stable | 70 | 0.40 |
| Cognitive Normal | 66 | 0.5 |
| Alzheimer's disease with dementia | 67 | 0.5 |

### Results

TABLE 2
Validation of p53 marker performance in diagnosis
of AD in asymptomatic and MCI stages: Results
by Deep sequencing mass spec method.

IP by Ab clone 2D3A8

| | P1 | P2 | P3 | P4 | P5 |
|---|---|---|---|---|---|
| Stable Cognitive Normal | 0 | 0 | 0 | 0 | 0 |
| Stable Alzheimer's disease with | 3 | 3 | 2 | 1 | 3 |
| dementia | | | | | |
| Preclinical (Cognitive Normal to | 3 | 3 | 0 | 3 | 0 |
| AD converters) | | | | | |
| MCI Stable | 0 | 0 | 0 | 1 | 0 |
| Prodromal (MCI converted to | 3 | 3 | 1 | 0 | 0 |
| Alzheimer's) | | | | | |

N of transiction 1 can be interpreted as “possible negative”, N of transiction = 2 is a possible; N of transictions = 0 is “negative” and N of transictions = 3 is positive.

### Example 2. Validation of a p53 Conformational Human Plasma Biomarker Specific to Alzheimer's Disease at the Pre-Clinical and Prodromal Stages of the Disease

Initial results from a longitudinal four-year study on clinical progression of cognitively normal subjects and people with mild cognitive impairment to probable AD measuring U-p53 signals measured by direct ELISA, shown very high predictive values for progression. Based on this finding, it was developed a highly accurate mass spectrometry method based on the identification of p53 peptides P1 and/or P2, for the diagnosis of Alzheimer's at the pre-clinical and prodromal stages of the disease and for the prognosis of cognitive decline. The maximum prognostic PPV for cognitive decline to Alzheimer's dementia was achieved in both subjects with MCI and asymptomatic. It is believed that both the diagnostic and prognostic performance of the p53 peptides, P1 and/or P2, is the highest that has been reported to date.

A specific mass spec method, which is the SRM (Selected Monitoring Reaction) method (by triple quadrupole mass spectrometer) was used to quantify both P1 and/or P2 in plasma samples from AD, asymptomatic, MCI subjects. By the triple quadrupole mass spectrometer which acts as essentially as a mass filter, it is possible to sequence the peptides by looking at the different transition peaks. Then, using a heavy labelled internal control for peptide 1 and 2, it is possible to quantitate those peptides with the maximum precision by Selected Monitoring Reaction (SRM) method.

### Prognostic Performance

It was evaluated the prognostic power of the biomarker in enriching for patients who are PiB+ but also will exhibit cognitive decline in 18 months from MCI subjects and in 18-72 months for asymptomatic subjects at presentation. This was performed, in order to assess the value of the biomarker in significantly enriching for cognitive decline as an endpoint in disease modifying trials in asymptomatic and patients with MCI. It was examined the prognostic power of P1 and P2, using the future conversion of clinical presentation. Statistical parameters able to describe the prognostic power of P1 and P2 are below reported:

TABLE 3
Prognostic Power of Asymptomatic subjects to AD diagnosis

| | Sequence | Sequence |
|---|---|---|
| | peptide 1 | Peptide 2 |
| Sensitivity | 72.2% | 89% |
| Specificity | 95.5% | 100% |
| PPV (positive predictive value) | 70.2% | 100% |
| NPV (negative predictive value) | 99.7% | 99.8% |
| AUC | 83% | 90.7% |
| P-value | 0.0022 | 0.0020 |

TABLE 4
Prognostic Power of MCI subjects to AD diagnosis

| | Sequence | Sequence |
|---|---|---|
| | peptide 1 | Peptide 2 |
| Sensitivity | 95% | 100% |
| Specificity | 95% | 100% |
| PPV (positive predictive value) | 88.2% | 100% |
| NPV (negative predictive value) | 98% | 100% |
| AUC | 96.3% | 100% |
| P-value | 0.0082 | 0.4724 |

### CONCLUSIONS

P1 and P2 can accurately enrich for patients that will exhibit cognitive decline from either an asymptomatic or MCI clinical presentation with very high specificity and sensitivity and thus enable the successful recruitment of patients for disease modifying drugs targeting the very early stages of the disease.

### Example 3

Differential Diagnosis of Alzheimer's Disease from Other Forms of Dementia

An analysis was also performed to validate previous data on the specificity of the U-P53 biomarker in Alzheimer's dementia. Well characterized Alzheimer's patients which were amyloid positive and had Alzheimer's dementia were compared with patients with clinical presentations of other dementias such as FTD (frontal-temporal dementia), Parkinson's, Lewi's Body and vascular dementia.

Plasma samples from OD (FTD, Vascular, Parkinson's, Lewi's Body) were collected at disease stage, anyway in a differential diagnosis, it is possible to establish that above a certain cutoff both peptide sequences showed a high specificity and sensitivity toward AD compared to OD.

Furthermore, it has been observed that AUC of both peptides is greater than 90%, it shows the high correlation between biomarker concentration and the AD diagnosis.

There is in literature an unmet need in differential diagnosis in Alzheimer's dementia is between: a) advanced FTD and advanced AD b) advanced LBD and AD as those can mimic clinical presentation is some cases. Anything that can exclude either AD or the other dementia has clinical value, thus high sensitivity of either dementia is enough to bring value.

TABLE 5
Performance measures for p53 peptides P1 and
P2 in differentiating Alzheimer's pathology
from other Dementias in subjects with dementia.

| | Sequence | Sequence |
|---|---|---|
| | peptide 1 | Peptide 2 |
| Sensitivity | 100% | 80% |
| Specificity | 85.7% | 85.7% |
| PPV (positive predictive value) | 87.5% | 84.8% |
| NPV (negative predictive value) | 100% | 81.1% |
| AUC | 94.3% | 90.7% |
| P-value | 0.0510 | 0.0171 |

### REFERENCES

1. Stanga, S. et al., 2010. Unfolded p53 in the pathogenesis of Alzheimer's disease: Is HIPK2 the link? Aging, 2(9), pp. 545-554.
2. Lanni, C. et al., 2007. Unfolded p53: A potential biomarker for Alzheimer's disease. In Journal of Alzheimer's Disease. pp. 93-99.
3. Uberti, D. et al., 2008. Conformationally altered p53: a putative peripheral marker for Alzheimer's disease. Neuro-degenerative diseases, 5(3-4), pp. 209-11.
4. Lanni, C. et al., 2008. Conformationally altered p53: a novel Alzheimer's disease marker?Molecular psychiatry, 13(6), pp. 641-7.
5. Lanni, C., Racchi, M., et al., 2010. Unfolded p53 in blood as a predictive signature signature of the transition from mild cognitive impairment to Alzheimer's disease. Journal of Alzheimer's disease: JAD, 20(1), pp. 97-104.
6. Buizza, L. et al., 2012. Conformational altered p53 as an early marker of oxidative stress in Alzheimer's disease. PloS one, 7(1), p.e29789
7. Arce-Varas N, et al. Comparison of extracellular and intracellular blood compartments highlights redox alterations in Alzheimer's and Mild Cognitive Impairment patients. Current Alzheimer Research 2017; 14(1): 112-122.
8. Uberti, D. et al., 2006. Identification of a mutant-like conformation of p53 in fibroblasts from sporadic Alzheimer's disease patients. Neurobiology of aging, 27(9), pp. 1193-201.
9. Lanni, C., Nardinocchi, L., et al., 2010. Homeodomain interacting protein kinase 2: a target for Alzheimer's beta amyloid leading to misfolded p53 and inappropriate cell survival. PloS one, 5(4), p.e10171.
10. Lanni, C. et al., 2008. Pharmacogenetics and Pharmagenomics, Trends in Normal and Pathological Aging Studies: Focus on p53. Current Pharmaceutical Design, 14(26), pp. 2665-2671.

1-20. (canceled)

21. A method of providing a quantified reaction mixture comprising one or more proteolytic peptides comprising P1 peptide (SEQ ID NO. 1 TEEENLR), the method comprising the steps of:
subjecting an isolated human p53 protein to protease digestion to generate one or more proteolytic peptides comprising said P1 peptide, wherein said p53 protein is isolated from a biological sample of said subject using a p53-specific antibody comprising a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15);
isolating said one or more proteolytic peptides;
providing a reaction mixture comprising said isolated P1 peptide; and
quantifying the amount of said P1 peptide in said reaction mixture, thereby providing said quantified reaction mixture.

22. The method of claim 21, further comprising comparing an amount of each of said one or more proteolytic peptides in said reaction mixture to an amount of each of one or more respective control peptides.

23. The method of claim 22, wherein said reaction mixture comprises a higher amount of said one or more proteolytic peptides relative to said respective control peptide.

24. The method of claim 21, wherein said heavy chain variable region comprises SEQ ID NO: 6 and said light chain variable region comprises SEQ ID NO: 7.

25. The method of claim 21, wherein the antibody comprises a heavy chain comprising SEQ ID NO:4 and a light chain comprising SEQ ID NO:5.

26. The method of claim 23, wherein said amount of said respective control peptide is 1 femtomole.

27. The method of claim 22, wherein said comparing is performed using mass spectrometry analysis.

28. The method of claim 27, wherein said mass spectrometry analysis comprises HPLC-mass spectrometry or deep sequence mass spectrometry.

29. The method of claim 22, wherein said control peptide is a labeled peptide.

30. The method of claim 29, wherein said labeled control peptide is internal to said reaction mixture and is represented as a value comprising its concentration in said reaction mixture.

31. The method of claim 22, wherein said comparing is performed using Selected Reaction Monitoring (SRM) and said SRM identifies and quantifies said one or more peptides comprised by said reaction mixture by comparing the same with said peptide control value.

32. The method of claim 21, wherein said biological sample is subjected to protein plasma depletion prior to said immunoprecipitation.

33. The method of claim 32, wherein said protein plasma depletion is accomplished by one or more of: HPLC, a chromatographic column, and/or chemical treatment of said biological sample.

34. The method of claim 22, wherein said comparing of said P1 peptide is relative to a control peptide P1.

35. The method of claim 22, wherein said one or more proteolytic peptides further comprises a P2 peptide (SEQ ID NO. 2 TEEENLRK[GG]K) and wherein said method further comprises quantifying the amount of said P2 peptide in said reaction mixture.

36. The method of claim 21, said proteolytic peptide in said reaction mixture is P1 peptide and said amount of P1 peptide is 0.05 fmol/40 μl to 6.70 fmol/40 μl.

37. The method of claim 1, said proteolytic peptide in said reaction mixture is P1 peptide and said amount of P1 peptide is 0.203 fmol/40 μl to 6.70 fmol/40 μl.

38. The method of claim 21, wherein said subject is cognitively normal (CN) or has mild cognitive impairment (MCI).

39. The method of claim 21, wherein said subject is suspected of having Alzheimer's disease (AD) or cognitive decline leading to AD.

40. The method of claim 21, wherein said biological sample comprises lymphocytes, plasma, serum or blood.

41. A composition comprising one or more proteolytic peptides comprising peptide P1 (SEQ ID NO. 1 TEEENLR) and peptide P2 (SEQ ID NO. 2 TEEENLRK[GG]K).

42. The composition of claim 41, wherein said one or more proteolytic peptides are proteolytic products of a protease digestion of an isolated human p53 protein, wherein said p53 protein is isolated from a biological sample of a human subject using a p53-specific antibody comprising a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15).

43. The composition of claim 22, wherein said antibody comprises a heavy chain variable region having SEQ ID NO: 8 and a light chain variable region having SEQ ID NO: 9.

44. The composition of claim 42, wherein said antibody comprises a heavy chain having SEQ ID NO: 6 and a light chain having SEQ ID NO: 7.

45. The composition of claim 42, wherein said biological sample is subjected to protein plasma depletion prior to said immunoprecipitation.

46. The composition of claim 45, wherein said protein plasma depletion is accomplished by one or more of: HPLC, a chromatographic column, and/or chemical treatment of said biological sample.

47. The composition of claim 42, wherein said biological sample comprises lymphocytes, plasma, serum or blood.

48. The composition of claim 42, wherein said sample is of a subject suspected of having MCI or cognitive decline leading to AD.

49. The composition of claim 42, wherein said protease is trypsin.

50. The composition of claim 42, further comprising one or more labeled control peptides comprising labeled P1 peptide and labeled P2 peptide.

51. A method for diagnosis of a subject as being cognitive normal (CN) or having mild cognitive impairment (MCI) or Alzheimer's disease (AD) or the prognosis of cognitive decline leading to dementia, the method comprising:
subjecting isolated p53 protein to protease digestion to generate one or more proteolytic peptides comprising P1 peptide, said peptide P1 having (SEQ ID NO. 1 TEEENLR) and said p53 protein being isolated from a biological sample of said subject using a p53-specific antibody comprising a heavy chain variable region comprising CDR1 (SEQ ID NO:10), CDR2 (SEQ ID NO: 11) and CDR3 (SEQ ID NO: 12) and a light chain variable region comprising CDR1 (SEQ ID NO:13), CDR2 (SEQ ID NO: 14) and CDR3 (SEQ ID NO: 15); and
comparing an amount of each of said one or more proteolytic peptides in a reaction mixture to an amount of a respective control peptide, wherein a higher amount of a said proteolytic peptide relative to said respective control peptide indicates Alzheimer's disease or cognitive decline leading to dementia in said subject.

52. The method of claim 51, wherein said amount of said control peptide is 1 femtomole.

53. The method of claim 51, wherein said comparing is performed using mass spectrometry analysis.

54. The method of claim 53, wherein said mass spectrometry analysis comprises HPLC-mass spectrometry or deep sequence mass spectrometry.

55. The method of claim 51, wherein said control peptide is a labeled peptide.

56. The method of claim 55, wherein said labeled control peptide is internal to said reaction mixture and is represented as a value comprising its concentration in said reaction mixture.

57. The method of claim 51, wherein said comparing is performed using Selected Reaction Monitoring (SRM).

58. The method of claim 56, wherein said comparing is performed using Selected Reaction Monitoring (SRM) and said SRM identifies and quantifies said one or more peptides comprised by said reaction mixture by comparing the same with said peptide control value.

59. The method of claim 51, wherein said biological sample is subjected to protein plasma depletion prior to said immunoprecipitation.

60. The method of claim 59, wherein said protein plasma depletion is accomplished by one or more of: HPLC, a chromatographic column, and/or chemical treatment of said biological sample.

61. The method of claim 51, wherein the method provides for the diagnosis of Alzheimer's disease in an asymptomatic subject or in a subject exhibiting mild cognitive impairment.

62. The method of claim 51, wherein the method provides for prognosis of cognitive decline in an asymptomatic subject or a subject exhibiting mild cognitive impairment.

63. The method of claim 61, wherein said prognosis is for cognitive decline leading to dementia.

64. The method of claim 63, wherein said prognosis is for said cognitive decline leading to said dementia leading to Alzheimer's disease.

65. The method of claim 51, wherein said comparing of said P1 peptide is relative to a control peptide P1.

66. The method of claim 51, wherein said one or more proteolytic peptides further comprises P2 peptide (SEQ ID NO. 2 TEEENLRK[GG]K) and wherein said method further comprises quantifying the amount of said P2 peptide in said reaction mixture.

67. The method of claim 51, wherein said antibody comprises a heavy chain variable region having SEQ ID NO: 8 and a light chain variable region having SEQ ID NO: 9.

68. The method of claim 51, wherein said antibody comprises a heavy chain having SEQ ID NO: 6 and a light chain having SEQ ID NO: 7.

69. The method of claim 51, a said proteolytic peptide in said reaction mixture is P1 peptide and said amount of P1 peptide is 0.05 fmol/40 μl to 6.70 fmol/40 μl.

70. The method of claim 51, a said proteolytic peptide in said reaction mixture is P1 peptide and said amount of P1 peptide is 0.203 fmol/40 μl to 6.70 fmol/40 μl.

71. The method of claim 69, wherein said subject is cognitively normal (CN) or has mild cognitive impairment (MCI).

72. The method of claim 70, wherein said subject has a prognosis of cognitive decline of Alzheimer's dementia.

73. The method of claim 51, further comprising the step of treating said subject for Alzheimer's disease or dementia.

74. The method of claim 51, wherein said biological sample comprises lymphocytes, plasma, serum or blood.
